(19) United States
(12) Patent Application Publication
(10) Pub. No.: US 2025/0204991 A1
(43) Pub. Date: Jun. 26, 2025

(54) SMART, VIDEO-BASED JOINT DISTRACTOR POSITIONING SYSTEM

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Craig A. Di Stefano, Andover, MA (US); Nathan Zamarripa, Kittery Point, ME (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/982,472

(22) Filed: Dec. 16, 2024

Related U.S. Application Data
(60) Provisional application No. 63/612,708, filed on Dec. 20, 2023.

(51) Int. Cl. A61B 34/20; A61B 17/68; A61B 34/10; A61B 90/00; G06T 7/11

(52) U.S. Cl. CPC A61B 34/20; A61B 17/68; A61B 34/10; A61B 90/39; G06T 7/11; A61B 2090/3916; A61B 2090/3937; G06T 2207/30008

(57) ABSTRACT

A processor is configured to execute instructions stored in memory to provide guidance during a surgical procedure. Executing the instructions causes the processor to obtain an image of patient anatomy including at least a femur and a pelvis, segment the image into a pelvis portion and a femur portion, generate a pre-operative plan for the surgical procedure using the segmented image, including calculating, using the segmented image, a surgical area of interest between the pelvis portion and the femur portion and a minimum distance between the femur and the pelvis within the surgical area of interest, and generate and provide, via a display, visual guidance for performing distraction during the surgical procedure, the visual guidance being generated based on the minimum distance.

### CROSS-REFERENCE TO RELATED APPLICATIONS

This application claims the benefit of U.S. Provisional Application No. 63/612,708, filed on Dec. 20, 2023. The entire disclosure of the application referenced above is incorporated herein by reference.

### FIELD

The present disclosure relates surgical navigation systems and methods, and more particularly to surgical navigation systems and methods for performing joint distraction.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Arthroscopic surgical procedures are minimally invasive surgical procedures in which access to the surgical site within the body is by way of small keyholes or ports through the patient's skin. The various tissues within the surgical site are visualized by way of an arthroscope placed through a port, and the internal scene is shown on an external display device. The tissue may be repaired or replaced through the same or additional ports. In computer-assisted surgical procedures (e.g., surgical procedures associated with a knee or knee joint, surgical procedures associated with a hip or hip joint, etc.), the location of various objects with the surgical site may be tracked relative to the bone by way of images captured by an arthroscope and a three-dimensional model of the bone.

### SUMMARY

An aspect of the disclosure includes a processor configured to execute instructions stored in memory to provide guidance during a surgical procedure. Executing the instructions causes the processor to obtain an image of patient anatomy including at least a femur and a pelvis, segment the image into a pelvis portion and a femur portion, generate a pre-operative plan for the surgical procedure using the segmented image, including calculating, using the segmented image, a surgical area of interest between the pelvis portion and the femur portion and a minimum distance between the femur and the pelvis within the surgical area of interest, and generate and provide, via a display, visual guidance for performing distraction during the surgical procedure, the visual guidance being generated based on the minimum distance.

In other aspects, a system is configured to perform functions corresponding to various methods described herein. In other aspects, various methods include steps corresponding to functions of systems described herein.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

For a detailed description of example embodiments, reference will now be made to the accompanying drawings in which:

FIG. 1 shows a surgical system in accordance with at least some embodiments;

FIG. 2 shows a conceptual drawing of a surgical site with various objects within the surgical site tracked, in accordance with at least some embodiments;

FIG. 3 shows a method in accordance with at least some embodiments;

FIG. 4 is an example video display showing portions of a femur and a bone fiducial during a registration procedure, in accordance with at least some embodiments;

FIG. 5 shows a method in accordance with at least some embodiments;

FIG. 6 shows an example hip distraction system in accordance with at least some embodiments;

FIGS. 7A and 7B show a segmented image used during hip distraction in accordance with at least some embodiments;

FIG. 8 shows an example femur including a fiducial marker used during hip distraction in accordance with at least some embodiments;

FIG. 9 shows an example method for performing hip distraction in accordance with at least some embodiments; and

FIG. 10 shows an example computer system or computing device configured to implement the various systems and methods of the present disclosure.

In the drawings, reference numbers may be reused to identify similar and/or identical elements.

### DEFINITIONS

Various terms are used to refer to particular system components. Different companies may refer to a component by different names-this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

Similarly, spatial and functional relationships between elements (for example, between device, modules, circuit elements, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. Nevertheless, this paragraph shall serve as antecedent basis in the claims for referencing any electrical connection as “directly coupled” for electrical connections shown in the drawing with no intervening element(s).

Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure. As one example, numerical values that are described as “approximate” or “approximately” as used herein may refer to a value within +/−5% of the stated value.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions. To be clear, an initial reference to “a [referent]”, and then a later reference for antecedent basis purposes to “the [referent]”, shall not obviate the fact the recited referent may be plural.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context. As used herein, the phrase at least one of A, B, and C should be construed to mean a logical (A OR B OR C), using a non-exclusive logical OR, and should not be construed to mean “at least one of A, at least one of B, and at least one of C.”

The terms “input” and “output” when used as nouns refer to connections (e.g., electrical, software) and/or signals, and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections and/or signals transmitted or received by those connections. In systems implemented in software, these “inputs” and “outputs” define parameters read by or written by, respectively, the instructions implementing the function. In examples where used in the context of user input, “input” may refer to actions of a user, interactions with input devices or interfaces by the user, etc.

“Controller,” “module,” or “circuitry” shall mean, alone or in combination, individual circuit components, an application specific integrated circuit (ASIC), a microcontroller with controlling software, a reduced-instruction-set computer (RISC) with controlling software, a digital signal processor (DSP), a processor with controlling software, a programmable logic device (PLD), a field programmable gate array (FPGA), or a programmable system-on-a-chip (PSOC), configured to read inputs and drive outputs responsive to the inputs.

As used to describe various surgical instruments or devices, such as a probe, the term “proximal” refers to a point or direction nearest a handle of the probe (e.g., a direction opposite the probe tip). Conversely, the term “distal” refers to a point or direction nearest the probe tip (e.g., a direction opposite the handle).

For the purposes of this disclosure, a non-transitory computer readable medium (or computer-readable storage medium/media) stores computer data, which data can include computer program code (or computer-executable instructions) that is executable by a computer, in machine-readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure, the term “server” should be understood to refer to a service point that provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Various surgical procedures may involve or require joint distraction. As used herein, the term “distraction” refers to a process in which bones or other anatomical structures within a joint are separated to form and/or increase a space or gap between the anatomical structures. The space between the anatomical structures facilitates performance of the surgical procedure within the joint. For example, hip distraction may be used to facilitate correction/repair of soft tissue and bone within a hip joint while minimizing interference with surrounding tissue. Hip distraction may be performed using a hip distraction system or assembly, which may be referred to as a hip distractor.

In some examples, hip distraction may be performed using one or more screws of a hip distraction system inserted into bone (e.g., into the femur). The hip joint can be slowly distracted by exerting force on the screws using visualization cues (e.g., from an arthroscopic camera) as well as feel from tactile feedback (e.g., tactile feedback provided by knobs or other components of the hip distraction system, by patient anatomy, surgical instruments, etc.), a technique that requires considerable experience.

Complications following hip arthroscopy and other procedures may include distraction-type injuries (e.g., injuries caused during/by hip distraction), which may occur in up to 7% of cases. Example distraction-type injuries include nerve injuries of the femoral, sciatic, or peroneal nerves due to an excessive traction force or a prolonged traction time.

Joint distraction systems and methods according to the present disclosure implement a smart, video-based surgical navigation and distractor/positioning system in which the surgeon follows on-screen, mixed reality guidance to achieve an exact joint (e.g., hip) distraction desired with sub-millimeter accuracy.

For example, surgical procedures may implement various systems and methods for operating and controlling surgical systems, such as arthroscopic video-based navigation systems and associated surgical tools or instruments. In some examples, navigation systems and tools (e.g., probes) may be used for registering a three-dimensional model of a rigid structure, such as bone, capturing images, and so on. These systems can be configured to identify surface features of a rigid structure visible in a video stream, and use the surface features to register a three-dimensional model for use in computer-assisted navigation of the surgical procedure. In some examples, the surface features are determined using touchless techniques based on a known or calculated motion of the camera. In other examples, the surface features are gathered using a touch probe that is not itself directly tracked; rather, the pose of the touch probe, and thus the locations of the distal tip of the touch probe touching the bone, may be determined by segmenting the frames of the video stream and pose estimation. In yet still further examples, the three-dimensional model may be registered by use of a patient-specific instrument that couples to the rigid structure in only one orientation; thus, a fiducial coupled to the patient-specific instrument, or in some cases the patient-specific instrument itself without a fiducial, may be used to register the three-dimensional bone model.

Various examples are described herein in the context of surgical procedures associated with the knee, hip, shoulder, ankle, etc. In this context, the rigid structure is bone, and the three-dimensional mode is a three-dimensional bone model. However, the techniques are applicable to any suitable rigid anatomical structure. Moreover, the various techniques may be applicable to many types of surgical procedures. Thus, the description and developmental context shall not be read as a limitation of the applicability of the teachings.

In some examples of a registration procedure, a user (e.g., a surgeon) probes an anatomical surface using a handheld probe. Collected points (e.g., a point cloud) are processed (e.g., using a machine learning algorithm) and matched to a bone model, such as a bone model created via a scan (e.g., a CT or MRI scan) or other technique. For example, the bone model is overlaid on top of a live arthroscopic video feed to provide an augmented or mixed reality visual representation of a surgical or anatomical site. In computer-assisted surgical procedures (e.g., procedures implementing surgical navigation techniques), surgical guidance may be provided in an image of a surgical site (e.g., within an image of the anatomy of a patient) to guide the surgeon throughout the surgical procedure.

Joint distraction systems and methods as described herein implement video-based surgical navigation techniques as described above for surgical procedures involving joint distraction. For example, for a surgical procedure for a hip joint according to the present disclosure, an imaging scan (e.g., a pre-operative imaging scan) may be performed on patient anatomy (e.g., for a hip procedure, an imaging scan of pelvis-to-toe anatomy). An image obtained from the imaging scan is segmented (e.g., for a hip procedure, into femur and pelvis portions) and can be stored in a memory/storage device, such as memory/storage of a surgical system, a cloud computing system, etc.

The segmented image may then be retrieved (e.g., using a tablet or other computing device), pre-and/or intra-operatively, for viewing/analysis by the surgical system, the surgeon, etc. For example, the segmented image can be used to formulate a pre-operative plan to distract the hip to provide access to a point or area of interest associated with performing the hip procedure. Formulating the pre-operative plan may determine, calculate, obtain, etc. various parameters associated with achieving a desired distraction profile, such as minimum distances (e.g., gaps) between anatomical structures in an area of interest, as well as forces, angles, etc. associated with achieving the minimum distances.

In an example, a fiducial marker (e.g., a marker with QR or other scannable codes) can be placed into a proximal femur to perform registration. On-screen guidance can be provided to achieve parameters outlined in the pre-operative plan and in accordance with information obtained using the fiducial marker (e.g., “articulate boot to position ‘C,’ “rotate distraction angle 40°,” “apply 7 mm of distraction on distraction knob,” etc.). By following the guidance, the results of the desired plan can be achieved without guesswork or other unpredictable variations. In this manner, joint distraction workflow is preserved but with mechanical adjustments being highly characterized and incremented into discrete adjustment points.

As one example, a video-based navigation system configured to implement the principles of the present disclosure may include devices such as an arthroscopic video camera head and console control unit (CCU), a video-based navigation (VBN) controller, a tablet, and/or various connected devices and instruments.

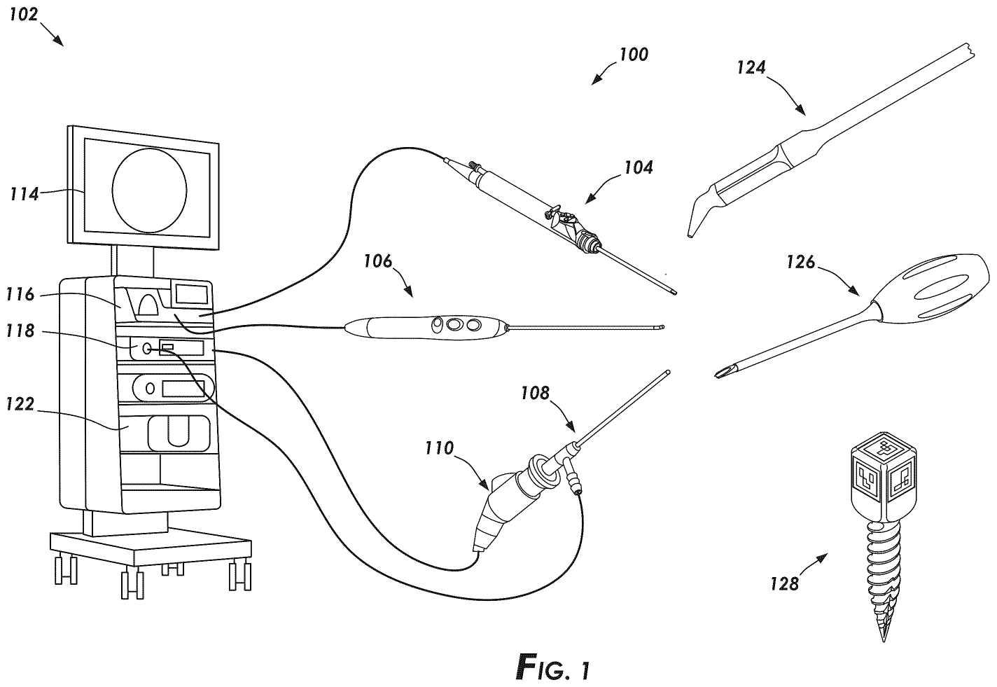

FIG. 1 shows an example surgical system (e.g., a system including or implementing an arthroscopic video-based navigation system) 100 in accordance with at least some embodiments of the present disclosure. In particular, the example surgical system 100 comprises a tower or device cart 102 and various tools or instruments, such as an example mechanical resection instrument 104, an example plasma-based ablation instrument (hereafter just ablation instrument 106), and an endoscope in the example form of an arthroscope 108 and attached camera head or camera 110. In the example systems, the arthroscope 108 may be a rigid device, unlike endoscopes for other procedures, such as upper-endoscopies. The device cart 102 may comprise a display device 114, a resection controller 116, and a camera control unit (CCU) together with an endoscopic light source and video (e.g., a VBN) controller 118. In example cases the combined CCU and video controller 118 not only provides light to the arthroscope 108 and displays images received from the camera 110, but also implements various additional aspects, such as registering a three-dimensional bone model with the bone visible in the video images, and providing computer-assisted navigation during the surgery. Thus, the combined CCU and video controller are hereafter referred to as surgical controller 118. In other cases, however, the CCU and video controller may be a separate and distinct system from the controller that handles registration and computer-assisted navigation, yet the separate devices would nevertheless be operationally coupled.

The example device cart 102 further includes a pump controller 122 (e.g., single or dual peristaltic pump). Fluidic connections of the mechanical resection instrument 104 and ablation instrument 106 to the pump controller 122 are not shown so as not to unduly complicate the figure. Similarly, fluidic connections between the pump controller 122 and the patient are not shown so as not to unduly complicate the figure. In the example system, both the mechanical resection instrument 104 and the ablation instrument 106 are coupled to the resection controller 116 being a dual-function controller. In other cases, however, there may be a mechanical resection controller separate and distinct from an ablation controller. The example devices and controllers associated with the device cart 102 are merely examples, and other examples include vacuum pumps, patient-positioning systems, robotic arms holding various instruments, ultrasonic cutting devices and related controllers, patient-positioning controllers, and robotic surgical systems.

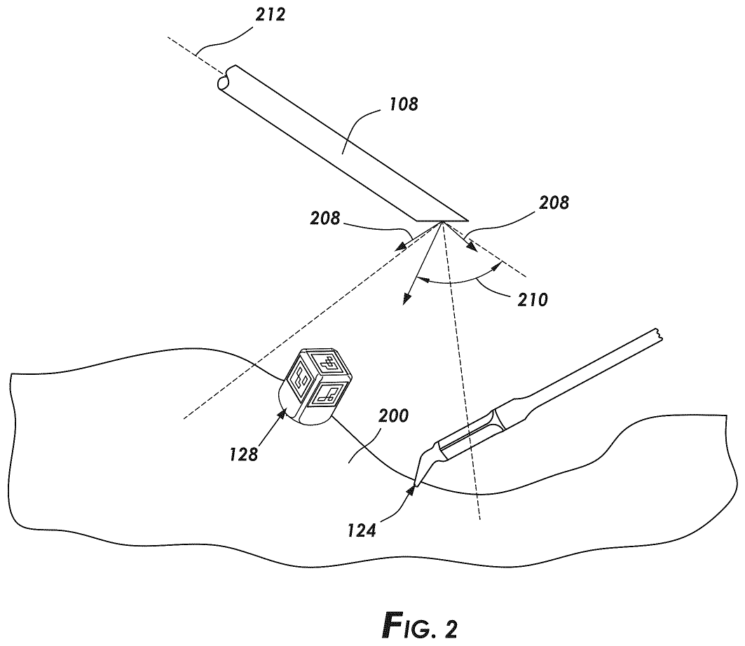

FIGS. 1 and 2 further show additional instruments that may be present during an arthroscopic surgical procedure. In particular, an example probe 124 (e.g., shown as a touch probe, but which may be a touchless probe in other examples), a drill guide or aimer 126, and a bone fiducial 128 are shown. The probe 124 may be used during the surgical procedure to provide information to the surgical controller 118, such as information to register a three-dimensional bone model to an underlying bone visible in images captured by the arthroscope 108 and camera head 110. In some surgical procedures, the aimer 126 may be used as a guide for placement and drilling with a drill wire to create an initial or pilot tunnel through the bone. The bone fiducial 128 may be affixed or rigidly attached to the bone and serve as an anchor location for the surgical controller 118 to know the orientation of the bone (e.g., after registration of a three-dimensional bone model). Additional tools and instruments may be present, such as the drill wire, various reamers for creating the throughbore and counterbore aspects of a tunnel through the bone, and various tools, such as for suturing and anchoring a graft. These additional tools and instruments are not shown so as not to further complicate the figure.

Example workflow for a surgical procedure is described below. While described with respect to an example anterior cruciate ligament repair procedure, the below techniques may also be performed for other types of surgical procedures, such as hip procedures or other procedures that include joint distraction. A surgical procedure may begin with a planning phase. An example procedure may start with imaging (e.g., X-ray imaging, computed tomography (CT), magnetic resonance imaging (MRI)) of the anatomy of the patient, including the relevant anatomy (e.g., for a knee procedure the lower portion of the femur, the upper portion of the tibia, and the articular cartilage; for a hip procedure, an upper portion of the femur, the acetabulum/hip joint, pelvis, etc.). The imaging may be preoperative imaging, hours or days before the intraoperative repair, or the imaging may take place within the surgical setting just prior to the intraoperative repair. The discussion that follows assumes MRI imaging, but again many different types of imaging may be used. The image slices from the MRI imaging can be segmented such that a volumetric model or three-dimensional model of the anatomy is created. Any suitable currently available, or after developed, segmentation technology may be used to create the three-dimensional model. More specifically to the example of anterior cruciate ligament repair, a three-dimensional bone model of the lower portion of the femur, including the femoral condyles, is created. Conversely, for a hip procedure, a three-dimensional model of the upper portion of the femur and at least a portion of the pelvis (e.g., the acetabulum) is created.

Using the three-dimensional bone model, an operative plan is created. For a knee procedure, the results of the planning may include: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia. Other surgical parameters may also be selected during the planning, such as tunnel throughbore diameters, tunnel counterbore diameters and depth, desired post-repair flexion, and the like, but those additional surgical parameters are omitted so as not to unduly complicate the specification.

Conversely, for a hip procedure, the results of the planning may include a three-dimensional bone model of the proximal end of the femur; a three-dimensional bone model for at least a portion of the pelvis/hip joint (e.g., a region of the pelvis corresponding to the acetabulum); a surgical area of interest within the hip joint; and parameters associated with achieving an amount of distraction in the surgical area of interest to provide sufficient access to the surgical area of interest. For example, example hip procedures may include, but are not limited to, labral repair, femoroacetabular impingement (FAI) debridement (e.g., removal of bone spurs/growths), cartilage repair, and synovectomy (e.g., removal of inflamed tissue). These example procedures typically require access to a specific surgical area of interest within the hip joint (i.e., in a specific area within an interface between the pelvis and the femoral head, such as an area around/surrounding a bone spur or growth, cartilage or tissue to be repaired or removed, etc.). Accordingly, the parameters may include ranges of values, minimum and/or maximum values, etc. required/recommended for providing access to the surgical area of interest within the hip joint. As one example, the parameters may include a minimum amount of distraction (e.g., a minimum space or gap) in an area around, centered on, etc. the surgical area of interest (e.g., a minimum gap at one or more entry/access points, for a surgical instrument, around a bone spur, bump or other anatomical feature associated with the surgical procedure).

The intraoperative aspects include steps and procedures for setting up the surgical system to perform the various repairs. It is noted, however, that some of the intraoperative aspects (e.g., optical system calibration) may take place before any ports or incisions are made through the patient's skin, and in fact before the patient is wheeled into the surgical room. Nevertheless, such steps and procedures may be considered intraoperative as they take place in the surgical setting and with the surgical equipment and instruments used to perform the actual repair.

An example procedure can be conducted arthroscopically and is computer-assisted in the sense that the surgical controller 118 is used for arthroscopic navigation within the surgical site. More particularly, in example systems the surgical controller 118 provides computer-assisted navigation during the procedure by tracking locations of various objects within the surgical site, such as the location of the bone within the three-dimensional coordinate space of the view of the arthroscope, and location of the various instruments within the three-dimensional coordinate space of the view of the arthroscope. A brief description of such tracking techniques is described below.

FIG. 2 shows a conceptual drawing of a surgical site with various objects (e.g., surgical instruments/tools) within the surgical site. In particular, visible in FIG. 2 is a distal end of the arthroscope 108, a portion of a bone 200 (e.g., femur), the bone fiducial 128 within the surgical site, and the probe 124.

The arthroscope 108 illuminates the surgical site with visible light. In the example of FIG. 2, the illumination is illustrated by arrows 208. The illumination provided to the surgical site is reflected by various objects and tissues within the surgical site, and the reflected light that returns to the distal end enters the arthroscope 108, propagates along an optical channel within the arthroscope 108, and is eventually incident upon a capture array within the camera 110 (FIG. 1). The images detected by the capture array within the camera 110 are sent electronically to the surgical controller 118 (FIG. 1) and displayed on the display device 114 (FIG. 1). In one example, the arthroscope 108 is monocular or has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 108, the camera 110, and the surgical controller 118 is provided with the arthroscope 108 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30° arthroscope the viewing direction forms a 30° angle to the longitudinal central axis of the arthroscope, the angle measured as an obtuse angle beyond the distal end of the arthroscope. In the example of FIG. 2, the view angle 210 of the arthroscope 108 forms a non-zero angle to the longitudinal central axis 212 of the arthroscope 108.

Still referring to FIG. 2, within the view of the arthroscope 108 is a portion of the bone 200 (in this example, within the intercondylar notch), along with the example bone fiducial 128, and the example probe 124. The example bone fiducial 128 is multi-faceted element, with each face or facet having a fiducial disposed or created thereon. However, the bone fiducial need not have multiple faces, and in fact may take any shape so long as that shape can be tracked within the video images. The bone fiducial, such as bone fiducial 128, may be attached to the bone 200 in any suitable form (e.g., via the screw portion of the bone fiducial 128 visible in FIG. 1). The patterns of the fiducials on each facet are designed to provide information regarding the orientation of the bone fiducial 128 in the three-dimensional coordinate space of the view of the arthroscope 108. More particularly, the pattern is selected such that the orientation of the bone fiducial 128 may be determined from images captured by the arthroscope 108 and attached camera (FIG. 1).

The probe 124 is also shown as partially visible within the view of the arthroscope 108. The probe 124 may be used, as discussed more below, to identify a plurality of surface features on the bone 200 as part of the registration of the bone 200 to the three-dimensional bone model. Alternatively, though not specifically shown, the aimer 126 (FIG. 1) may be used as the device to help with the registration process. In some cases the probe 124 and/or the aimer 126 may carry their own, unique fiducials, such that their respective poses may be calculated from the one or more fiducial present in the video stream. However, in other cases, and as shown, the medical instrument used to help with registration of the three-dimensional bone model, be it the probe 124, the aimer 126, or any other suitable medical device, may omit carrying fiducials. Stated otherwise, in such examples the medical instrument has no fiducial markings. In such cases, the pose of the medical instrument may be determined by a machine learning model, discussed in more detail below.

The images captured by the arthroscope 108 and attached camera are subject to optical distortion in many forms. For example, the visual field between distal end of the arthroscope 108 and the bone 200 within the surgical site is filled with fluid, such as bodily fluids and saline used to distend the joint. Many arthroscopes have one or more lenses at the distal end that widen the field of view, and the wider field of view causes a “fish eye” effect in the captured images. Further, the optical elements within the arthroscope (e.g., rod lenses) may have optical aberrations inherent to the manufacturing and/or assembly process. Further still, the camera may have various optical elements for focusing the images received onto the capture array, and the various optical elements may have aberrations inherent to the manufacturing and/or assembly process. In example systems, prior to use within each surgical procedure, the endoscopic optical system is calibrated to account for the various optical distortions. The calibration creates a characterization function that characterizes the optical distortion, and further analysis of the frames of the video stream may be, prior to further analysis, compensated using the characterization function.

The next example step in the intraoperative procedure is the registration of the bone model created during the planning stage. During the intraoperative repair, the three-dimensional bone model is obtained by or provided to the surgical controller 118. Again using the example of anterior cruciate ligament repair, and specifically computer-assisted navigation for tunnel paths through the femur, the three-dimensional bone model of the lower portion of the femur is obtained by or provided to the surgical controller 118. Thus, the surgical controller 118 receives the three-dimensional bone model, and assuming the arthroscope 108 is inserted into the knee by way of a port through the patient's skin, the surgical controller 118 also receives video images of a portion of the lower end of the femur. In order to relate the three-dimensional bone model to the images received by way of the arthroscope 108 and camera 110, the surgical controller 118 registers the three-dimensional bone model to the images of the femur received by way of the arthroscope 108 and camera 110.

In order to perform the registration, and in accordance with example methods, the bone fiducial 128 is attached to the femur. The bone fiducial placement is such that the bone fiducial is within the field of view of the arthroscope 108. In examples for knee procedures, the bone fiducial 128 is placed within the intercondylar notch superior to the expected location of the tunnel through lateral condyle. Conversely, in examples for hip procedures, the bone fiducial 128 is placed on the femoral head. To relate or register bone visible in the video images to the three-dimensional bone model, the surgical controller 118 (FIG. 1) is provided or determines a plurality of surface features of an outer surface of the bone. Identifying the surface features may take several forms, including a touch-based registration using the probe 124 without a carried fiducial, a touchless registration technique in which the surface features are identified after resolving the motion of the arthroscope 108 and camera relative to the bone fiducial 128, and a third technique in which uses a patient-specific instrument.

In the example touch-based registration, the surgeon may touch a plurality of locations using the probe 124 (FIG. 1). In some cases, particularly when portions of the outer surface of the bone are exposed to view, receiving the plurality of surface features of the outer surface of the bone may involve the surgeon “painting” the outer surface of the bone. “Painting” is a term of art that does not involve application of color or pigment, but instead implies motion of the probe 124 when the distal end of the probe 124 is touching bone. In this example, the probe 124 does not carry or have a fiducial visible to the arthroscope 108 and the camera 110. It follows that the pose of the probe 124 and the location of the distal tip of the probe 124 needs to be determined in order to gather the surface features for purposes of registering the three-dimensional bone model.

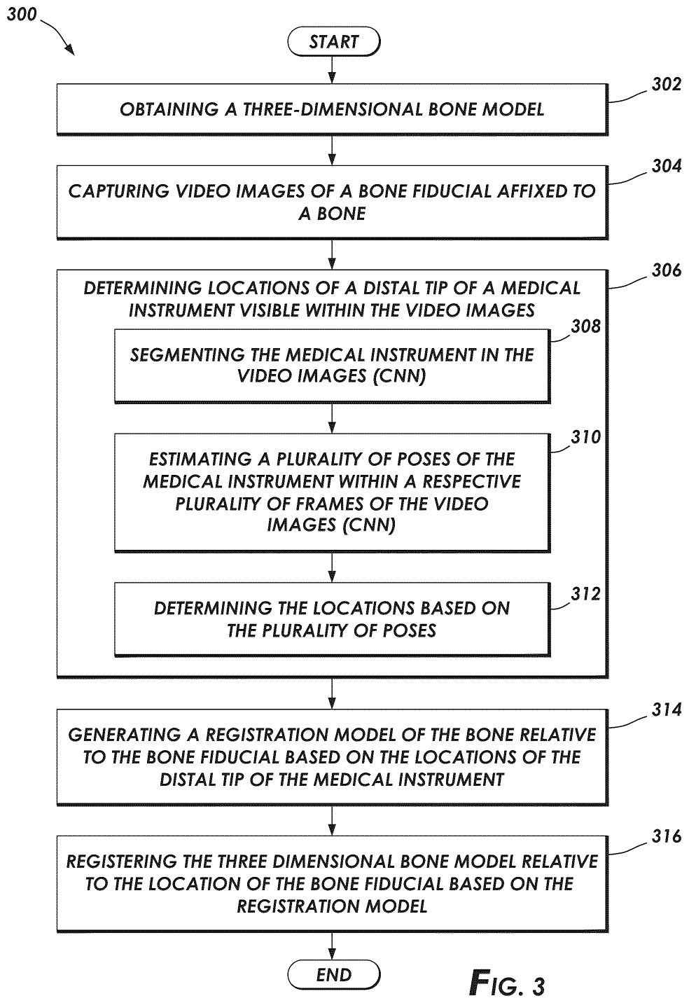

FIG. 3 shows a method 300 in accordance with at least some embodiments of the present disclosure. The example method 300 may be implemented in software within a computer system, such as the surgical controller 118. In particular, the example method 300 comprises obtaining a three-dimensional bone model (block 302). That is to say, in the example method 300, what is obtained is the three-dimensional bone model that may be created by segmenting a plurality of non-invasive images (e.g., CT, MRI) taken preoperatively or intraoperatively. With the bone segmented from or within the images, the three-dimensional bone model may be created. The three-dimensional bone may take any suitable form, such as a computer-aided design (CAD) model, a point cloud of data points with respect to an arbitrary origin, or a parametric representation of a surface expressed using analytical mathematical equations. Thus, the three-dimensional bone model is defined with respect to the origin and in any suitable an orthogonal basis.

The next step in the example method 300 is capturing video images of the bone fiducial attached to the bone (block 304). The capturing is performed intraoperatively. In an example, the capturing of video images is by way of the arthroscope 108 and camera 110. Other endoscopes may be used, such as endoscopes in which the capture array resides at the distal end of the device (e.g., chip-on-the-tip devices). However, in open procedures where the skin is cut and pulled away, exposing the bone to the open air, the capturing may be by any suitable camera device, such as one or both cameras of a stereoscopic camera system, or a portable computing device, such as a tablet or smart-phone device. The video images may be provided to the surgical controller 118 in any suitable form.

The next step in the example method 300 is determining locations of a distal tip of the medical instrument visible within the video images (block 306), where the distal tip is touching the bone in at least some of the frames of the video images, and the medical instrument does not have a fiducial. Determining the locations of the distal tip of the medical instrument may take any suitable form. In one example, determining the locations may include segmenting the medical instrument in the frames of the video images (block 308). The segmenting may take any suitable form, such as applying the video images to a segmentation machine learning algorithm. The segmentation machine learning algorithm may take any suitable form, such as neural network or convolution neural network trained with a training data set showing the medical instrument in a plurality of known orientations. The segmentation machine learning algorithm may produce segmented video images where the medical instrument is identified or highlighted in some way (e.g., box, brightness increased, other objects removed).

With the segmented video images, the example method 300 may estimate a plurality of poses of the medical instrument within a respective plurality of frames of the video images (block 310). The estimating the poses may take any suitable form, such as applying the video images to a pose machine learning algorithm. The pose machine learning algorithm may take any suitable form, such as neural network or convolution neural network trained to perform six-dimensional pose estimation. The resultant of the pose machine learning algorithm may be, for at least some of the frames of the video image, an estimated pose of the medical instrument in the reference frame of the video images and/or in the reference frame provided by the bone fiducial. That is, the resultant of the pose machine learning algorithm may be a plurality of poses, one pose each for at least some of the frames of the segmented video images. While in many cases a pose may be determined for each frame, in other cases it may not be possible to make a pose estimation for at least some frame because of video quality issues, such as motion blur caused by electronic shutter operation.

The next step in the example method 300 is determining the locations based on the plurality of poses (block 312). In particular, for each frame for which a pose can be estimated, based on a model of the medical device the location of the distal tip can be determined in the reference frame of the video images and/or the bone fiducial. Thus, the resultant is a set of locations that, at least some of which, represent locations of the outer surface of the bone.

FIG. 3 shows an example three-step process for determining the locations of the distal tip of the medial instrument. However, the method 300 is merely an example, and many variations are possible. For example, a single machine learning model, such as a convolution neural network, may be set up and trained to perform all three steps as a single overall process, though there may be many hidden layers of the convolution neural network. That is, the convolution neural network may segment the medical instrument, perform the six-dimensional pose estimation, and determine the location of the distal tip in each frame. The training data set in such a situation would include a data set in which each frame has the medical device segmented, the six-dimensional pose identified, and the location of the distal tip identified. The output of the determining step 306 may be a segmented video stream distinct from the video images captured at step 304. In such cases, the later method steps may use both segmented video stream and the video images to perform the further tasks. In other cases, the location information may be combined with the video images, such as being embedded in the video images, or added as metadata to each frame of the video images.

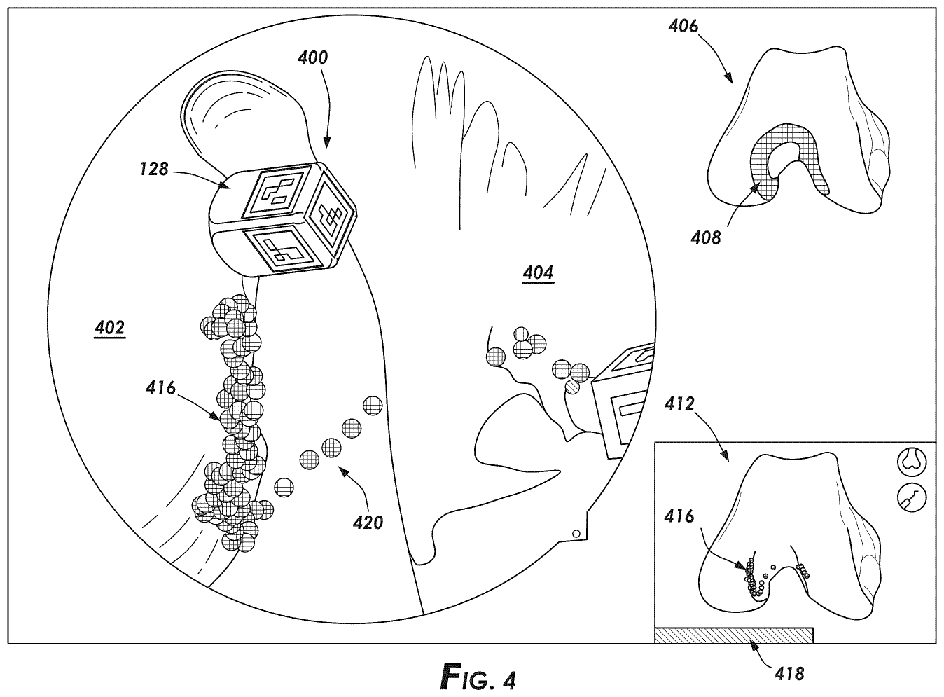

FIG. 4 is an example video display showing portions of a femur and a bone fiducial during a registration procedure. Although described with respect to a distal end of a femur, the principles and techniques described and shown in FIG. 4 can be applied to other anatomical structures/procedures, such as a femoral head for hip procedures as described herein. The display may be shown, for example, on the display device 114 associated with the device cart 102, or any other suitable location. In particular, visible in the main part of the display of FIG. 4 is an intercondylar notch 400, a portion of the lateral condyle 402, a portion the medial condyle 404, and the example bone fiducial 128. Shown in the upper right corner of the example display is a depiction of the bone, which may be a rendering 406 of the bone created from the three-dimensional bone model. Shown on the rendering 406 is a recommended area 408, the recommended area 408 being portions of the surface of the bone to be “painted” as part of the registration process. Shown in the lower right corner of the example display is a depiction of the bone, which again may be a rendering 412 of the bone created from the three-dimensional bone model. Shown on the rendering 412 are a plurality of surface features 416 on the bone model that have been identified as part of the registration process. Further shown in the lower right corner of the example display is progress indicator 418, showing the progress of providing and receiving of locations on the bone. The example progress indicator 418 is a horizontal bar having a length that is proportional to the number of locations received, but any suitable graphic or numerical display showing progress may be used (e.g., 0% to 100%).

Referring to both the main display and the lower right rendering, as the surgeon touches the outer surface of the bone within the images captured by the arthroscope 108 and camera 110, the surgical controller 118 receives the surface features on the bone, and may display each location both within the main display as dots or locations 416, and within the rendering shown in the lower right corner. More specifically, the example surgical controller 118 overlays indications of identified surface features 416 on the display of the images captured by the arthroscope 108 and camera 110, and in the example case shown, also overlays indications of identified surface features 416 on the rendering 412 of the bone model. Moreover, as the number of identified locations 416 increases, the surgical controller 118 also updates the progress indicator 418.

Still referring to FIG. 4, in spite of the diligence of the surgeon, not all locations identified by the surgical controller 118 based on the surgeon's movement of the probe 124 result in valid locations on the surface of the bone. In the example of FIG. 4, as the surgeon moves the probe 124 from the inside surface of the lateral condyle 102 to the inside surface of the medial condyle 104, the surgical controller 118, based on the example six-dimensional pose estimation, receives several locations 420 that likely represent locations at which the distal end of the probe 124 was not in contact with the bone.

With reference to FIG. 3, the plurality of surface features 416 may be, or the example surgical controller 118 may generate, a registration model relative to the bone fiducial 128 (block 314). The registration model may take any suitable form, such as a computer-aided design (CAD) model or point cloud of data points in any suitable orthogonal basis. The registration model, regardless of the form, may have fewer overall data points or less “structure” than the bone model created by the non-invasive computer imaging (e.g., MRI). However, the goal of the registration model is to provide the basis for the coordinate transforms and scaling used to correlate the bone model to the registration model and relative to the bone fiducial 128. Thus, the next step in the example method 300 is registering the bone model relative to the location of the bone fiducial based on the registration model (block 316). Registration may conceptually involve testing a plurality of coordinate transformations and scaling values to find a correlation that has a sufficiently high correlation or confidence factor. Once a correlation is found with the sufficiently high confidence factor, the bone model is said to be registered to the location of the bone fiducial. Thereafter, the example registration method 300 may end (block 318); however, the surgical controller 118 may then use the registered bone model to provide computer-assisted navigation regarding a procedure involving the bone.

In the examples discussed to this point, registration of the bone model involves a touch-based registration technique using the probe 124 without a carried fiducial. However, other registration techniques are possible, such as a touchless registration technique. The example touchless registration technique again relies on placement of the bone fiducial 128. As before, when the viewing direction of the arthroscope 108 is relatively constant, the bone fiducial may have fewer faces with respective fiducials. Once placed, the bone fiducial 128 represents a fixed location on the outer surface of the bone in the view of the arthroscope 108, even as the position of the arthroscope 108 is moved and changed relative to the bone fiducial 128. Again, in order to relate or register the bone visible in the video images to the three-dimensional bone model, the surgical controller 118 (FIG. 1) determines a plurality of surface features of an outer surface of the bone, and in this example determining the plurality of surface features is based on a touchless registration technique in which the surface features are identified based on motion of the arthroscope 108 and camera 110 relative to the bone fiducial 128.

Another technique for registering the bone model to the bone uses a patient-specific instrument. In both touch-based and touchless registration techniques, a registration model is created, and the registration model is used to register the bone model to the bone visible in the video images. Conceptually, the registration model is used to determine a coordinate transformation and scaling to align the bone model to the actual bone. However, if the orientation of the bone in the video images is known or can be determined, use of the registration model may be omitted, and instead the coordinate transformations and scaling may be calculated directly.

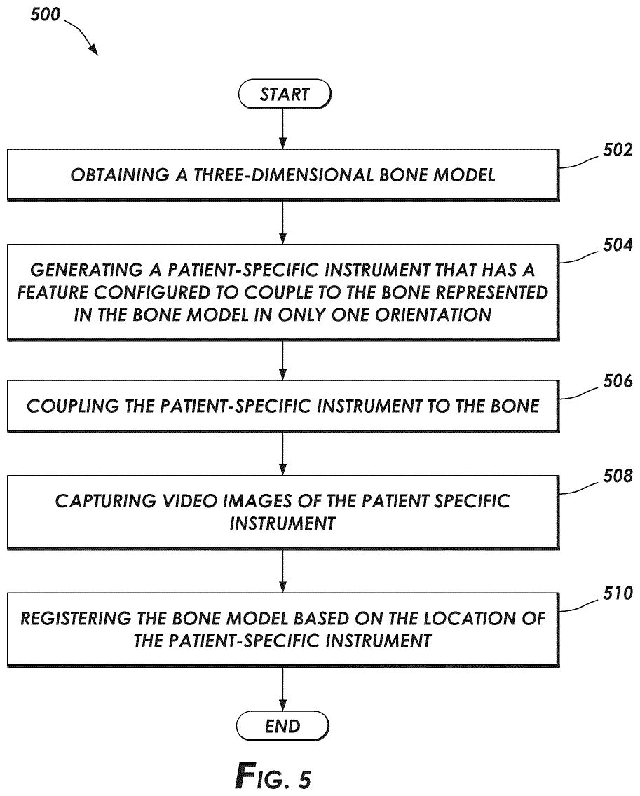

FIG. 5 shows a method 500 in accordance with at least some embodiments. The example method may be implemented in software within one or more computer systems, such as, in part, the surgical controller 118. In particular, the example method 500 comprises obtaining a three-dimensional bone model (block 502). In the patient-specific instrument registration technique, what is obtained is the three-dimensional bone model that may be created by segmenting a plurality of non-invasive images (e.g., MRI) taken preoperatively or intraoperatively.

The method 500 further includes generating a patient-specific instrument that has a feature designed to couple to the bone represented in the bone model in only one orientation (block 504). Generating the patient-specific instrument may first involve selecting a location at which the patient-specific instrument will attach. For example, a device or computer system may analyze the bone model and select the attachment location. In various examples, the attachment location may be a unique location in the sense that, if a patient-specific instrument is made to couple to the unique location, the patient-specific instrument will not couple to the bone at any other location. In the example case of an anterior cruciate ligament repair, the location selected may be at or near the upper or superior portion on the intercondylar notch. If the bone model shows another location with a unique feature, such as a bone spur or other raised or sunken surface anomaly, such a unique location may be selected as the attachment location for the patient-specific instrument. For example, for hip procedures, the location may be selected based on a location, within the hip joint, of a bone spur or other anatomical feature associated with the hip procedure.

Moreover, forming the patient-specific instrument may take any suitable form. In one example, a device or computer system may directly print, such as using a 3D printer, the patient-specific instrument. In other cases, the device or computer system may print a model of the attachment location, and the model may then become the mold for creating the patient-specific instrument. For example, the model may be the mold for an injection-molded plastic or casting technique. In some examples, the patient-specific instrument carries one or more fiducials, but as mentioned above, in other cases the patient-specific instrument may itself be tracked and thus carry no fiducials.

The method 500 further includes coupling the patient-specific instrument to the bone, in some cases the patient-specific instrument having the fiducial coupled to an exterior surface (block 506). As described above, the attachment location for the patient-specific instrument can be selected to be unique such that the patient-specific instrument couples to the bone in only one location and in only one orientation. In the example case of an arthroscopic procedure, the patient-specific instrument may be inserted arthroscopically. That is, the attachment location may be selected such that a physical size of the patient-specific instrument enables insertion through the ports in the patient's skin. In other cases, the patient-specific instrument may be made or constructed of a flexible material that enables the patient-specific instrument to deform for insertion in the surgical site, yet return to the predetermined shape for coupling to the attachment location. However, in open procedures where the skin is cut and pulled away, exposing the bone to the open air, the patient-specific instrument may be a rigid device with fewer size restrictions.

The method 500 further includes capturing video images of the patient-specific instrument (block 508). Here again, the capturing may be performed intraoperatively. In the example case of an arthroscopic anterior cruciate ligament repair, the capturing of video images is by the surgical controller 118 by way of arthroscope 108 and camera 110. However, in open procedures where the skin is cut and pulled away, exposing the bone to the open air, the capturing may be by any suitable camera device, such as one or both cameras of a stereoscopic camera systems, or a portable computing device, such as a tablet or smart-phone device. In such cases, the video images may be provided to the surgical controller 118 in any suitable form.

The example method 500 further includes registering the bone model based on the location of the patient-specific instrument (block 510). That is, given that the patient-specific instrument couples to the bone at only one location and in only one orientation, the location and orientation of the patient-specific instrument is directly related to the location and origination of the bone, and thus the coordinate transformations and scaling for the registration may be calculated directly. Thereafter, the example method 500 may end; however, the surgical controller 118 may then use the registered bone model to provide computer-assisted navigation regarding a surgical task or surgical procedure involving the bone.

For example, with the registered bone model the surgical controller 118 may provide guidance regarding a surgical task of a surgical procedure. The specific guidance is dependent upon the surgical procedure being performed and the stage of the surgical procedure. A non-exhaustive list of guidance comprises: changing a drill path entry point; changing a drill path exit point; aligning an aimer along a planned drill path; showing location at which to cut and/or resect the bone; reaming the bone by a certain depth along a certain direction; placing a device (suture, anchor or other) at a certain location; placing a suture at a certain location; placing an anchor at a certain location; showing regions of the bone to touch and/or avoid; and identifying regions and/or landmarks of the anatomy. In yet still other cases, the guidance may include highlighting within a version of the video images displayed on a display device, which can be the arthroscopic display or a see-through display, or by communicating to a virtual reality device or a robotic tool.

Joint distraction systems and methods according to the principles of the present disclosure are configured to implement video-based surgical navigation techniques (e.g., such as described above in FIGS. 1-5) and joint distraction techniques as described below in more detail. Although described with respect to hip distraction for hip procedures, these techniques may be implemented to perform distraction for other types of joints/procedures.

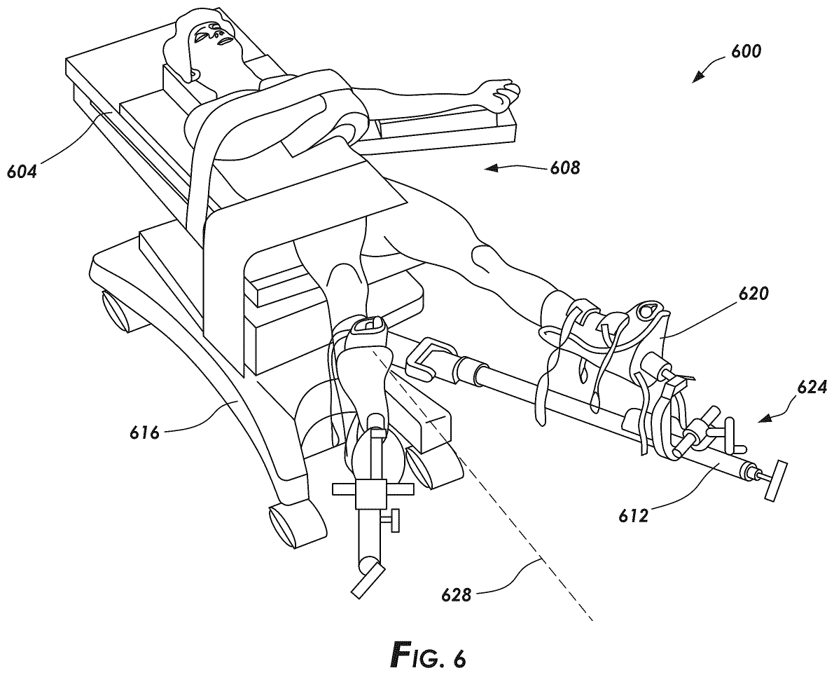

FIG. 6 shows one example hip distraction system or distractor 600 for use with the joint distraction systems and methods of the present disclosure. For example, the hip distraction system 600 (or other suitable distraction system or assembly, which may differ for respective types of joint procedures) may be configured to interface/communicate with a video-based surgical navigation system, such as systems/devices associated with the system 100 (e.g., via various sensors, connected tools or instruments, cameras or other imaging devices, etc.).

Generally, the hip distraction system 600 may include a table, platform, or other surface 604 configured to support a patient 608 during a hip procedure. Adjustable leg spars 612 extend from a base 616 of the hip distraction system 600. Each of the leg spars 612 is configured to attach to, retain, and provide traction for a respective leg of the patient 608. For example, feet/lower legs of the patient 608 are secured within respective traction boots 620, which are in turn connected to the leg spars 612. As shown at 624, the leg spars 612 include various actuators (knobs, handles, etc.) that can be actuated to adjust the position of the legs of the patient 608, apply traction (e.g., increase and decrease an amount of traction), etc. In this manner, the hip distraction system 600 can be used by the surgeon to adjust leg positions and angles (e.g., relative angles of the femur of the patient to the pelvis/hip joint, which may correspond to an angle of the leg spar 612 to a central axis of the hip distraction system 600 as shown at 628), increase and decrease an amount of force applied at various angles, and increase and decrease an amount of distraction. In some examples, the actuators 624 may be adjusted manually (e.g., by the surgeon). In other examples, various actuators may be configured for automatic/electronic adjustment (e.g., responsive to control signals provided by the system 100).

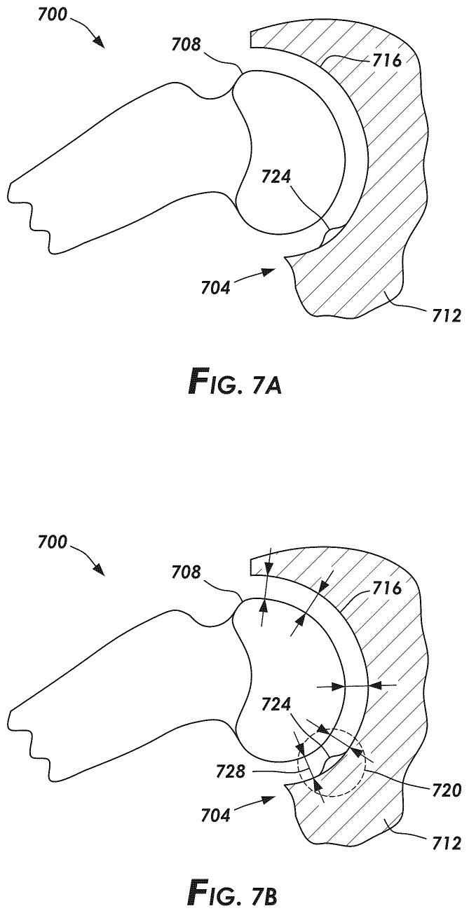

FIG. 7A shows an example segmented image 700 obtained using an imaging scan (e.g., a pre-operative imaging scan, such as a CT scan) performed on patient anatomy. As shown, the segmented image 700 obtained from the imaging scan is segmented into femur and pelvis portions and can be stored in a memory/storage device, such as memory/storage of a surgical system, a cloud computing system, etc. For example, for a hip procedure, the imaging scan can be performed on pelvis-to-toe anatomy of the patient, resulting in an image including anatomical structures of a hip joint 704. The structures of the hip joint 704 may include, but are not limited to, a proximal end of a femur 708 (e.g., a femoral head), a portion of a pelvis 712, and an acetabulum 716 (i.e., a socket of the pelvis 712 configured to receive and retain the femoral head).

The segmented image 700 may then be retrieved (e.g., using a tablet or other computing device), pre- and/or intra-operatively, for viewing/analysis by the surgical system 100, the surgeon, etc. For example, the segmented image 700 can be used to formulate a pre-operative plan to distract the hip joint 704 to provide access to a point or area of interest associated with performing the hip procedure as shown in FIG. 7B. Formulating the pre-operative plan may include determining, calculating, obtaining, etc. various parameters associated with achieving a desired distraction profile, such as minimum distances (e.g., gaps) between anatomical structures in an area of interest, as well as forces, angles, etc. associated with achieving the minimum distances.

As an example, the hip joint 704 may include a surgical area of interest 720 corresponding to a target surgical area or feature of the hip procedure. In this example, the surgical area of interest 720 corresponds to a bone spur 724 or bump to be removed or treated during the hip procedure. In other examples, the surgical area of interest 720 may correspond to cartilage, tissue, or other anatomical features within the hip joint 704. The surgical area of interest 720 may be selected/identified by the surgeon (e.g., using the surgical system 100 or other system or device with a user interface enabling interaction with the segmented image 700), by the system 100 (e.g., using machine learning or other Al systems/techniques), etc.

In some examples, the surgeon identifies/selects a target anatomical feature of the hip procedure (e.g., the bone spur 724, such as by selecting the bone spur 724 on a display or other interface displaying the image 700) and the area of interest 720. In other examples, the surgeon identifies the bone spur 724 and the system 100 determines the area of interest 720 based on the identified bone spur 724 (e.g., by highlighting/defining an area around the bone spur 724, such as an area having a minimum radius relative to the bone spur 724). In still other examples, the system 100 identifies the bone spur 724 and identifies the area of interest 720. Although shown as a circle or sphere, the area of interest 720 can be other shapes, including non-symmetrical or irregular shapes, depending on patient anatomy, type of procedure, etc.

A distance analysis is then performed (e.g., by the system 100) to calculate one or more minimum distances 728 required between surfaces of the femur 708 and the pelvis 712, within the area of interest 720, to provide access to the bone spur 724. For example, the minimum distances 728 may correspond to distances between the femur 708 and the pelvis 712 at a plurality of points within the area of interest 720. In an example, the minimum distances may correspond to a single minimum distance (i.e., a single minimum distance for all points within the area of interest 720). In other examples, the minimum distances 728 may vary for different points within the area of interest 720.

In some examples, the minimum distance 728 may correspond to a predetermined minimum distance (e.g., a baseline minimum distance) for a given type of procedure, location within the hip joint 704, etc. In other examples, the minimum distance 728 may be calculated further based on a type or size of an instrument/tool used in the procedure (e.g., type of burr, size of burr, etc.). For example, the minimum distance 728 may be calculated based on some tolerance or offset amount greater than a relevant size or dimension (e.g., diameter, width, thickness) of an end of an instrument requiring access to the area of interest 720. In one example, the instrument may be auto-detected by the system 100 (e.g., upon connection of the instrument to the system 100, selection/initiation of a surgical procedure, etc.). In another example, an identifier or indication of the instrument or characteristics/dimensions of the instrument may be input by the surgeon.

The system 100 calculates an amount of distraction (or a range of distraction) required based on the one or more minimum distances 728. In some examples, the amount of direction may correspond directly to (e.g. may be approximately equal to) the minimum distances 728. In other examples, the amount of distraction may correspond to a predetermined or calculated amount greater than the minimum distances 728. The amount of distraction may be defined based on a single parameter/value (e.g., a minimum distance to be achieved within the area of interest 720) or a range of values (e.g., a minimum distance required to provide access and a maximum distance not to be exceeded).

In some examples, calculating the distraction amount may include calculating distraction distances as well as one or more other distraction parameters, such as forces (e.g., a minimum force required to achieve the minimum distance, a maximum force not to be exceeded, etc.) and/or distraction angles (e.g., one or more angles indicating positions of the leg/femur 708 relative to the pelvis 712 or a central axis of the patient, positions of the spars 612 relative to the axis 628, etc.).

In some examples, calculating the distraction amount may include calculating two or more angles/positions, a range of angles, two or more ranges of angles (e.g., non-contiguous ranges of angles), etc. For example, based on patient anatomy, the procedure being performed, the instrument being used, etc., it may be possible to achieve the minimum distance at multiple different angles or within different ranges of angles. As one example, the minimum distance may be achievable a first range of angles/positions (e.g., with a distraction angle between 32 and 36 degrees). As another example, the minimum distance may be achievable in both the first range of angles and a second range of angles (e.g., with a distraction angle between 40 and 44 degrees). In these examples, respective forces and/or distraction amounts required to achieve the minimum distance within each of these ranges can be calculated. In this manner, an angle or range of angles corresponding to a least amount of force, minimum distraction amount, etc. (i.e., relative to other angles or range of angles) required to achieve the minimum distance can be selected or recommended by the system 100.

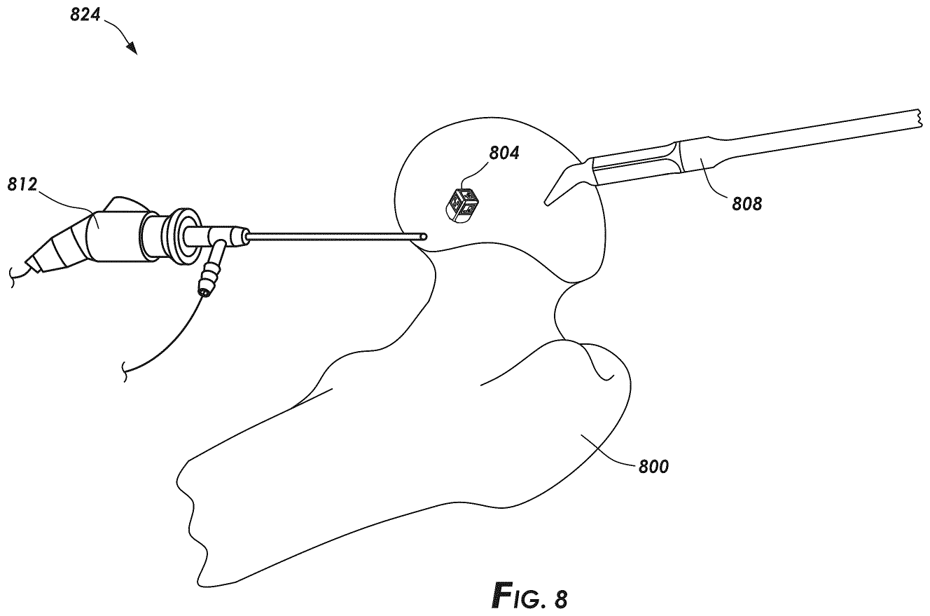

In an example shown in FIG. 8, a proximal end of a femur 800 includes a fiducial instrument/marker 804 (e.g., a marker with QR or other scannable codes). For example, the fiducial marker 804 can be placed in the proximal head of the femur 800 to perform registration (e.g., registration using a probe 808 as described herein). Subsequent to registration, the fiducial marker 804 is used during distraction techniques performed in accordance with the principles of the present disclosure. For example, during distraction, a scope 812 or other imaging device may be used to track the fiducial marker 804 as the femur 800 is distracted relative to the pelvis/hip joint. As one example, the scope 812 is positioned within the hip joint during distraction. In this manner, various parameters associated with the position, distraction amount, distraction angle, etc. of the femur can be tracked by the system 100, as well as measurements/calculations of various distances between the femur 800 and the pelvis within the area of interest 720 as described above in FIGS. 6A and 6B.

Using various parameters/measurements obtained in this manner, the system 100 can provide on-screen, audio, and/or other guidance to the surgeon (and/or automatically perform adjustments to the hip distraction system 600) to achieve the parameters (e.g., one or more minimum distances) determined/defined in the pre-operative plan and in accordance with information obtained using the fiducial marker. For example, for a selected distraction angle, the system 100 may provide instructions including, but not limited to: instructions to move a boot (e.g., the boot 620) to a specific position; rotate the spar 612 to a specific distraction angle; and apply a specific amount of distraction (e.g., with respect to distance and/or force), such as by manipulation one or more of the actuators 624.

The instructions/guidance may include visual guidance, such as a 3D or other graphical representation of the system 600 and/or the patient 608 overlaid with specific instructions, desired parameters (e.g., a desired distraction angle or amount), current parameters (e.g., a current distraction angle or amount), etc. The distraction amount may correspond to the actual minimum distance required or an additional distraction distance required, or to fixed distraction steps/adjustment amounts specific to the system 600. The visual guidance may further include the segmented image 700 and/or a real-time model of patient anatomy showing the hip joint, the distance between the femur and the pelvis within the area of interest 720, etc. Further, the guidance and/or parameters may be updated during distraction (e.g., in real-time). For example, recommended/desired angles, distraction amounts, etc. may vary during actual manipulation of the femur/hip joint. As one example, measurements obtained using the scope 812, marker 804, etc. may indicate that the minimum distance was achieved prior to a certain angle, distraction force or amount, and/or other parameter was reached using the system 600. As such, the system 100 may provide visual guidance, instructions, alerts, etc. indicating that no additional distraction is required.

Accordingly, by following the guidance as described above, the results of the pre-operative plan can be achieved based on the one or more minimum distances required to provide access for and perform the procedure.

Although described with respect to the system 600, the principles described herein may also be applied to other types of positioning systems/devices, such as a limb positioning systems for arthrotomy or arthroscopy procedures.

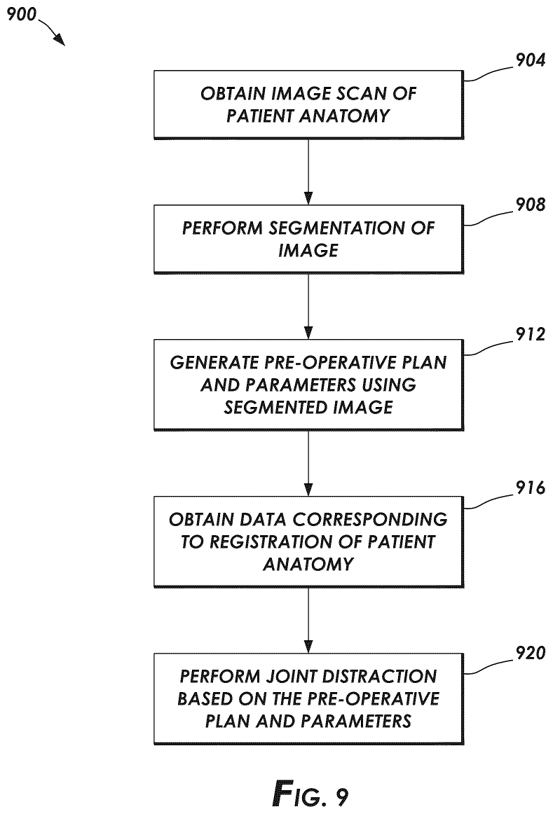

FIG. 9 shows an example method 900 for performing distraction in according with the principles of the present disclosure. As described, the method 900 may be performed by one or more processing devices or processors, computing devices, etc., such as the system 100 or another computing device executing instructions stored in memory. One or more steps of the method 900 may be omitted in various examples, and/or may be performed in a different sequence than shown in FIG. 9. The steps may be performed sequentially or non-sequentially, two or more steps may be performed concurrently, etc.

At 904, the method 900 includes obtaining an image scan of patient anatomy, such as by performing a pre-operative imaging scan (e.g., a CT scan), retrieving the stored image scan from memory, etc. At 908, the method 900 includes performing segmentation on the image to obtain a segmented image. For example, for a hip procedure, performing segmentation may include segmenting the image into femur and pelvis portions.

At 912, the method 900 includes generating a pre-operative plan using the segmented image. As used herein, “generating a pre-operative plan” refers to calculating various parameters or values, ranges of parameters, etc. required to provide access to an anatomical feature, area, or area of interest to perform the hip procedure. For example, generating the pre-operative plan may include, but is not limited to: identifying the anatomical feature (e.g., a bone spur); calculating a surgical area of interest around the anatomical feature; calculating one or more minimum distances, within the area of interest, required to provide access to the anatomical feature; and calculating one or more parameters required/recommended for achieving the one or more minimum distances. As one example, the one or more minimum distances are calculated based in part on dimensions of an instrument being used in the procedure. Calculating the one or more parameters may include calculating one or more distraction angles or ranges, calculating distraction amounts/distances, and/or calculating one or more forces.

At 916, the method 900 includes obtaining registration data corresponding to registration of patient anatomy (e.g., data such as digital indicators of patient anatomy obtained as described herein, such as by attaching a fiducial marker to patient anatomy and registering a plurality of points using a probe or other instrument).

At 920, the method 900 includes performing joint distraction based on the parameters determined/calculated for the pre-operative plan, the registration data, and real-time imaging information (e.g., information obtained using a scope or other imaging device during distraction). For example, performing joint distraction may include performing adjustments to the hip distraction system (e.g., manually and/or by controlling various actuators, motors, etc.) in response to guidance/instructions generated by the system 100. In an example, performing joint distraction may include displaying or otherwise providing guidance or instructions, based on the one or more minimum distances and real-time imaging data, for adjusting the hip distraction system as described herein.

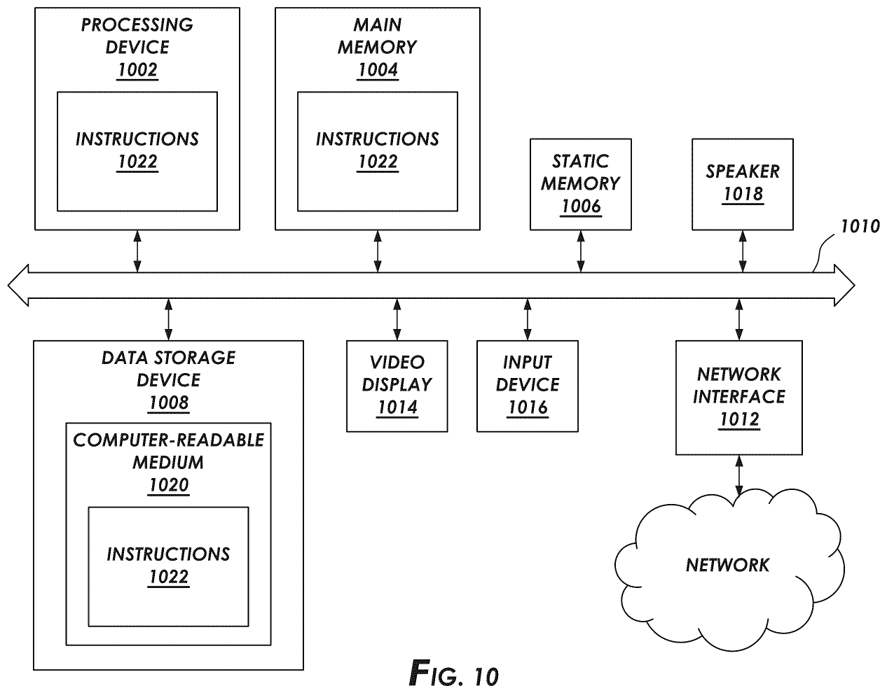

FIG. 10 shows an example computer system or computing device 1000 configured to implement the various systems and methods of the present disclosure. In one example, the computer system 1000 may correspond to one or more computing devices of the system 100, the surgical controller 118, a device that creates a patient-specific instrument, a tablet device within the surgical room, the controller 812, or any other system that implements any or all the various methods discussed in this specification. For example, the computer system 1000 may be configured to implement all or portions of the method 900. The computer system 1000 may be connected (e.g., networked) to other computer systems in a local-area network (LAN), an intranet, and/or an extranet (e.g., device cart 102 network), or at certain times the Internet (e.g., when not in use in a surgical procedure). The computer system 1000 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1000 includes a processing device 1002, a main memory 1004 (e.g., read-only memory (ROM), flash memory, dynamic random access memory (DRAM) such as synchronous DRAM (SDRAM)), a static memory 1006 (e.g., flash memory, static random access memory (SRAM)), and a data storage device 1008, which communicate with each other via a bus 1010.

Processing device 1002 represents one or more general-purpose processing devices such as a microprocessor, central processing unit, or the like. More particularly, the processing device 1002 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1002 may also be one or more special-purpose processing devices such as an application specific integrated circuit (ASIC), a field programmable gate array (FPGA), a digital signal processor (DSP), network processor, or the like. The processing device 1002 is configured to execute instructions for performing any of the operations and steps discussed herein. Once programmed with specific instructions, the processing device 1002, and thus the entire computer system 1000, becomes a special-purpose device, such as the surgical controller 118.

The computer system 1000 may further include a network interface device 1012 for communicating with any suitable network (e.g., the device cart 102 network). The computer system 1000 also may include a video display 1014 (e.g., the display device 114), one or more input devices 1016 (e.g., a microphone, a keyboard, and/or a mouse), and one or more speakers 1018. In one illustrative example, the video display 1014 and the input device(s) 1016 may be combined into a single component or device (e.g., an LCD touch screen).

The data storage device 1008 may include a computer-readable storage medium 1020 on which the instructions 1022 (e.g., implementing any methods and any functions performed by any device and/or component depicted described herein) embodying any one or more of the methodologies or functions described herein is stored. The instructions 1022 may also reside, completely or at least partially, within the main memory 1004 and/or within the processing device 1002 during execution thereof by the computer system 1000. As such, the main memory 1004 and the processing device 1002 also constitute computer-readable media. In certain cases, the instructions 1022 may further be transmitted or received over a network via the network interface device 1012.

While the computer-readable storage medium 1020 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

What is claimed is:

1. A processor configured to execute instructions stored in memory to provide guidance during a surgical procedure, wherein executing the instructions causes the processor to:
obtain an image of patient anatomy including at least a femur and a pelvis;
segment the image into a pelvis portion and a femur portion;
generate a pre-operative plan for the surgical procedure using the segmented image, wherein generating the pre-operative plan includes calculating, using the segmented image, (i) a surgical area of interest between the pelvis portion and the femur portion and (ii) a minimum distance between the femur and the pelvis within the surgical area of interest; and
generate and provide, via a display, visual guidance for performing distraction during the surgical procedure, wherein the visual guidance is generated based on the minimum distance.

2. The processor of claim 1, wherein the visual guidance includes instructions associated with at least one of an angle of the femur relative to the pelvis, a distraction amount, and a force to be applied to the patient anatomy.

3. The processor of claim 1, wherein the executing the instructions further causes the processor to generate the visual guidance based on detection of a fiducial marker arranged within the patient anatomy.

4. The processor of claim 1, wherein the executing the instructions further causes the processor to capture the image of the patient anatomy and store the image.

5. The processor of claim 4, wherein the executing the instructions further causes the processor to obtain the image by retrieving the image from memory.

6. The processor of claim 1, wherein calculating the surgical area of interest includes calculating the surgical area of interest based on a location of an anatomical feature of at least one of the femur and the pelvis.

7. The processor of claim 1, wherein calculating the minimum distance includes calculating the minimum distance based on a surgical instrument to be used during the surgical procedure.

8. The processor of claim 1, wherein calculating the minimum distance includes calculating a plurality of minimum distances at respective points within the surgical area of interest.

9. The processor of claim 1, wherein executing the instructions further causes the processor to control an actuator of a hip distraction system.

10. A method for providing guidance during a surgical procedure, the method comprising, using one or more computing devices:
obtaining an image of patient anatomy including at least a femur and a pelvis;
segmenting the image into a pelvis portion and a femur portion;
generating a pre-operative plan for the surgical procedure using the segmented image, wherein generating the pre-operative plan includes calculating, using the segmented image, (i) a surgical area of interest between the pelvis portion and the femur portion and (ii) a minimum distance between the femur and the pelvis within the surgical area of interest; and
generating and providing, via a display, visual guidance for performing distraction during the surgical procedure, wherein the visual guidance is generated based on the minimum distance.

11. The method of claim 10, wherein the visual guidance includes instructions associated with at least one of an angle of the femur relative to the pelvis, a distraction amount, and a force to be applied to the patient anatomy.

12. The method of claim 10, further comprising generating the visual guidance based on detection of a fiducial marker arranged within the patient anatomy.

13. The method of claim 10, further comprising capturing the image of the patient anatomy and storing the image.

14. The method of claim 13, further comprising obtaining the image by retrieving the image from memory.

15. The method of claim 10, wherein calculating the surgical area of interest includes calculating the surgical area of interest based on a location of an anatomical feature of at least one of the femur and the pelvis.

16. The method of claim 10, wherein calculating the minimum distance includes calculating the minimum distance based on a surgical instrument to be used during the surgical procedure.

17. The method of claim 10, wherein calculating the minimum distance includes calculating a plurality of minimum distances at respective points within the surgical area of interest.

18. The method of claim 10, further comprising controlling an actuator of a hip distraction system.

19. A joint distraction system, comprising:
one or more computing devices configured to
obtain an image of patient anatomy including at least a femur and a pelvis;
segment the image into a pelvis portion and a femur portion;
generate a pre-operative plan for a surgical procedure using the segmented image, wherein generating the pre-operative plan includes calculating, using the segmented image, (i) a surgical area of interest between the pelvis portion and the femur portion and (ii) a minimum distance between the femur and the pelvis within the surgical area of interest; and
generate and provide, via a display, visual guidance for performing distraction during the surgical procedure, wherein the visual guidance is generated based on the minimum distance.

20. The joint distraction system of claim 19, wherein the one or more computing devices are further configured to receive, from an imagine device, registration data corresponding to the patient anatomy and provide the visual guidance further based on the registration data.
